(19) United States
(12) Patent Application Publication
(10) Pub. No.: US 2002/0082471 A1
(43) Pub. Date: Jun. 27, 2002

(54) Pressure based spontaneous inflation inhibitor in a pump for an inflatable prosthesis

(75) Inventors: Gregory J. Henkel, Chanhassen, MN (US); Larry E. Waldack, Bloomington, MN (US); Charles C. Kuyava, Eden Prairie, MN (US); Randy L. Morningstar, Brooklyn Park, MN (US)

Correspondence Address: OPPENHEIMER WOLFF & DONNELLY LLP, 840 NEWPORT CENTER DRIVE, SUITE 700, NEWPORT BEACH, CA, 92660, US

(73) Assignee: American Medical Systems, Inc.

(21) Appl. No.: 09/749,292

(22) Filed: Dec. 27, 2000

(51) Int. Cl. A61F 2/04

(52) U.S. Cl. 600/39; 623/23.68; 623/23.67

(57) ABSTRACT

A pump assembly for a penile implant is provided having a mechanism which prevents spontaneous inflation of the cylinders implanted within the patient. The preventative mechanism uses overpressure generated by the reservoir during unintentional compression to effectively seal the cylinders from unintended fluid flow. The prevention mechanism itself creates all necessary forces to prevent the undesired fluid flow to the cylinders. This is accomplished by incorporating appropriate mechanisms within the pump itself.

### CROSS REFERENCE TO RELATED APPLICATIONS

This application is related to patent applications entitled “DIAPHRAGM BASED SPONTANEOUS INFLATION INHIBITOR IN A PUMP FOR AN INFLATABLE PROSTHESIS,” “SWITCH BASED SPONTANEOUS INFLATION INHIBITOR IN A PUMP FOR AN INFLATABLE PROSTHESIS,” and “PENILE PROSTHESIS PUMP WITH RELIEF VALVE” which were filed concurrently herewith.

### BACKGROUND OF THE INVENTION

This invention generally relates to a pump for inflating a prosthesis and more particularly to a pump and valve assembly including pressure based mechanisms that inhibit spontaneous inflation of the prosthesis.

One common treatment for male erectile dysfunction is the implantation of a penile prosthesis. Such prosthesis typically includes a pair of inflatable cylinders, which are fluidly connected to a fluid (typically liquid) reservoir via a pump and valve assembly. The two cylinders are normally implanted into the corpus cavernosae of the patient and the reservoir is typically implanted in the patient's abdomen. The pump assembly is implanted in the scrotum. During use, the patient actuates the pump and fluid is transferred from the reservoir through the pump and into the cylinders. This results in the inflation of the cylinders and thereby produces the desired penis rigidity for a normal erection. Then, when the patient desires to deflate the cylinders, a valve assembly within the pump is actuated in a manner such that the fluid in the cylinders is released back into the reservoir. This deflation then returns the penis to a flaccid state.

With inflatable penile prostheses of current designs, spontaneous inflation of the cylinders is known to occasionally occur due to inadvertent compression of the reservoir, resulting in the undesired introduction of fluid into the cylinders. Such inadvertent inflation can be uncomfortable and embarrassing for the patient. This undesirable condition is further described below with reference to a particular prosthetic design.

With reference to FIG. 1, a known pump and valve assembly 8 for use in a penile prosthesis includes a fluid input 10 that is coupled at one end to a reservoir (not shown) and to a housing 12 at its opposite end. Also connected to the housing 12 is a fluid output 14 which, in turn, is connected at its other end to a pair of cylinders (not shown). Linking the fluid input 10 and the fluid output 14 to each other is a common passageway 33, which itself contains a valve assembly that is described in greater detail below. Common passageway 33 is also in fluid communication with a pump bulb 18 that is used to move fluid from the reservoir (not shown) to the cylinders (not shown) in order to inflate the cylinders. The valve assembly located within common passageway 33 includes a reservoir poppet 20 which is biased against a valve seat 24 by a spring 28 and a cylinder poppet 22 which is biased against a valve seat 26 by a spring 30. The springs 28 and 30 are sized so as to keep the reservoir poppet 20 and the cylinder poppet 22 biased against each respective valve seat 24 and 26 under the loads that are encountered when the reservoir is pressurized to typical abdominal pressures.

When the patient wishes to inflate the cylinders, pump bulb 18 is squeezed so as to force fluid from the pump bulb 18 into the common passageway 33. The resulting fluid flow serves to reinforce the force from the spring 28 urging the reservoir poppet 20 against valve seal 24 while at the same time causing compression of the spring 30, and thereby opening cylinder poppet 22. As a result, the fluid travels out through fluid output 14 and into the respective cylinders.

When the patient releases the pump bulb 18 a vacuum is created, thus pulling the poppet 22 back against valve seat 26 (aided by spring 30) and simultaneously pulling the reservoir poppet 20 away from its valve seat 24, against the spring 28. As a result, fluid from the reservoir is thus allowed to flow through the fluid input 10 and into the common passageway 33 passing around the reservoir poppet 20 and into the vacuous pump bulb 18. Once the pump bulb 18 has been filled, the negative pressure is eliminated and the reservoir poppet 20 returns to its normal position. This pumping action of the pump bulb 18 and valve assembly is repeated until the cylinders are fully inflated.

To deflate the cylinders, the patient grips the housing 12 and compresses it along the axis of reservoir poppet 20 and cylinder poppet 22 in a manner such that the wall 13 of the housing 12 contacts the protruding end 21 of the reservoir poppet 20 and forces the reservoir poppet 20 away from valve seat 24. This movement, in turn, causes the reservoir poppet 20 to contact cylinder poppet 22 and force cylinder poppet 22 away from valve seat 26. As a result, both poppets 20 and 22 are moved away from their valve seats 24 and 26 and fluid moves out of the cylinders, through the fluid output 14, through common passageway 33, through the fluid input 10 and back into the reservoir.

Although the springs 28 and 30 are sized to provide sufficient tension to keep poppets 20 and 22 firmly abutted against valve seats 24 and 26 under normal reservoir pressures, it is possible that pressure that exceeds the force provided by the springs could be exerted upon the reservoir during heightened physical activity or movement by the patient. Such excessive pressure on the reservoir may overcome the resistance of the spring-biased poppets 20 and 22 and thereby cause a spontaneous inflation of the cylinders. After implantation, encapsulation or calcification of the reservoir may occur. This encapsulation or calcification of the reservoir. In particular, the encapsulation could lead to a more snugly enclosed reservoir, thus increasing the likelihood of spontaneous inflation.

In previous attempts to reduce or eliminate the occurrence of spontaneous inflation, different types of spontaneous inflation preventing valves have been introduced into the pump and valve assembly. Such previous valves are intended to permit the positive flow of fluid to the cylinders only in those circumstances when the patient has forcibly manipulated the valve.

Although such previous valve designs reduce the frequency of spontaneous inflation, several drawbacks do exist. For example, such valves are typically complex, requiring two-handed operation, which is a serious drawback to elderly or severely ill patients. Some spontaneous inflation preventing valves also require the application of excessive force in order to manipulate the valves; which may be too demanding for some patients. Furthermore, such valve designs may cause patient discomfort due to the valve size or shape, because of increase in the overall volume of the implant within the patient. This increased size can also lead to interference with the patient's normal bodily functions. Finally, such previous valve designs typically add undesirable cost to the device as well as increase the complexity of the surgical implantation procedure. As such, there exists a need to provide a prosthetic penile implant having a spontaneous inflation prevention mechanism that addresses the problems encountered in the prior art.

### BRIEF SUMMARY OF THE INVENTION

The present invention includes a penile pump having a dual poppet arrangement wherein the poppets act as check valves or flow valves. Each poppet is spring-biased against a valve seat, and under normal circumstances, only allows positive fluid flow when a pump bulb is operated, thus causing an increase in fluid pressure that is transferred to the inflatable cylinders. To prevent spontaneous inflation when an overpressurization occurs in the reservoir, the same reservoir pressure is utilized to seal the fluid output against itself or to seal one or both of the poppets against the valve seat. Thus, the fluid is prevented from reaching the cylinders and creating a spontaneous inflation. When the movement or activity generating the overpressure in the reservoir is released, the system should return to equilibrium. Even if overpressurization of the reservoir is occurring, the pressure generated by compressing the pump bulb will far exceed the level of overpressure. Thus, the poppets will open in the normal way, allowing fluid to flow to the cylinders. The use of the overpressure in the reservoir itself to prevent fluid flow to the cylinders can occur in a variety of formats.

In still another embodiment, the reservoir poppet is actually coupled to an outer wall defining a portion of the fluid input. When an overpressurization in the reservoir occurs, this outer wall is forced to expand which simultaneously causes the reservoir poppet to be pulled firmly against the valve seat. This effectively prevents fluid flow from reaching the cylinders and causing a spontaneous inflation.

In yet another embodiment of the present invention, the valve seat is provided with a flexible valve that cooperates with the first poppet to prevent spontaneous inflation, which could be caused by excessive pressure in the reservoir. Specifically, pressure in the reservoir and associated valve input is presented to the flexible valve and thus causing the valve to be further forced against the poppet, thus sealing off the input. When inflation is desired however, the negative pressure pulling the first poppet away from the valve seat will allow the desired fluid flow.

In yet still another embodiment, a tapered poppet us utilized in conjunction with a tapered valve seat. Each of these tapers do not exactly match each other, thus providing variable reactions to pressure signals.

In a further embodiment, a section of the reservoir poppet protrudes into the reservoir chamber. This protruding section of the reservoir poppet is coupled to the outer wall of the reservoir chamber. The poppet is coupled to the wall with a connecting spring that permits relative movement between the poppet and the outer wall. The tension of the spring is selected so that it approximates the forces generated by pressurized fluid acting on the wall of the reservoir chamber. However, the spring force is not so great as to prevent the vacuum generated by the pump bulb from opening the poppet. Thus, when the pump bulb is compressed and released, the vacuum forces generated are sufficient to unseat to the reservoir poppet despite its connection to the outer reservoir chamber wall.

In yet still a further embodiment, a relatively large and powerful biasing spring is coupled with the reservoir poppet to exert a relatively large force against the reservoir poppet forcing it into a sealing or closed position. Due to the strong biasing forces of the spring, overpressurization forces generated in the reservoir chamber are insufficient to unseat the reservoir poppet. Simply using such a spring will make it difficult for the vacuum forces generated by compression of the pump bulb to unseat the reservoir poppet. To eliminate this problem, the face of the reservoir poppet, which forms a fluid-tight seal when the reservoir poppet is in a closed position, is made relatively large. That is, the diameter of the face approaches the diameter of the chamber containing the reservoir poppet. Thus, the vacuum forces generated will act over a larger surface area thereby exerting a larger degree of force, which permits the unseating of the reservoir poppet despite the opposing force of the biasing spring.

Because it is difficult to fabricate a housing having a planar wall that interacts with the planar poppet face to form a sufficiently fluid-tight seal, the portion of the housing holding the reservoir poppet contains a pair of spaced lip seals. The position of the lip seal serves two distinct purposes. The first is to prevent fluid pressure generated during over pressurization of the reservoir from engaging a large portion of the poppet face, which would in effect defeat the added strength provided by the biasing spring. The outer seal is also provided so that when a vacuum force is generated, the vacuum cannot act on the front surface of the poppet face that would, in effect, hold the reservoir poppet in a closed position.

In still another embodiment of the present invention, the reservoir poppet is configured with a throughbore at a rear portion of the reservoir poppet that is in fluid communication with a passageway and an outlet adjacent to the cylinder poppet. A sliding valve seal is positioned over this section of the reservoir poppet. The sliding valve seal is held against the back wall of the chamber by a spring positioned between the front face of the sliding valve seal and the back face of the suction poppet valve seal. The arrangement of the valve sleeve on the rear of the reservoir poppet is such that fluid is only able to flow through the throughbore and out of the outlet when the valve sleeve is positioned near the rear of the chamber and the front face of the reservoir poppet is firmly seated. In a reservoir overpressurization situation, the valve sleeve is again pressed against the rear of the chamber. However, the reservoir poppet is also forced backwards into the chamber, forcing the throughbore to be occluded by the valve sleeve. This prevents fluid from flowing towards the cylinder poppet, which could ultimately lead to spontaneous inflation.

In still yet another embodiment, the portion of the housing between the cylinder poppet and the reservoir chamber has been modified. In addition, the reservoir poppet is provided with a unique configuration to interact with the housing structure. The reservoir poppet has a face, similar to the other embodiments, that is spring biased towards a matching valve seat. An annular ring is molded into the housing just behind (towards the cylinder poppet) the valve seat and is sized to interact with the face.

The pump assembly of this embodiment has two states, activated and deactivated. In the activated state, the reservoir poppet is positioned so that the face is between the annular ring and the valve seat. When so positioned, the pump assembly functions as previously described with reference to the other embodiments. A compression of the pump bulb force the face against the valve seat and causes the cylinder poppet to open. A release of the pump bulb generates a vacuum which removes the reservoir poppet face from the valve seat and allows fluid to flow from the reservoir and into the pump bulb. Thus, the activated state is used when actively inflating the cylinders and while it is desired to maintain the cylinders in an inflated state.

In the deactivated state, the reservoir poppet is positioned so that the face moves through the annular ring. In this position, the face will be between the cylinder poppet and the annular ring and the reservoir poppet spring will bias the face so that it abuts the annular ring. In other words, the face is displaced from the valve seat, and a gap exists between the valve seat and the annular ring. The stem of the reservoir poppet extends from the face towards the cylinder poppet. The stem is a cylindrical member having a generally V-shaped groove extending about its circumference near the middle of the stem. The stem interacts with a flexible conical lip seal molded within the housing. When in the activated state, the conical lip seal is positioned near the V-shaped groove so that fluid flow is essentially unhindered. When in the deactivated state, the conical lip seal is caused to engage the cylindrical portion of the stem. Thus, a fluid tight seal can be formed.

When in the deactivated state, the reservoir poppet can be moved to engage and release the cylinder poppet, leading to a deflation of the cylinders. During this time, the conical lip seal continues to be located near the cylindrical portion of the stem; however, the flexible nature of the conical lip seal allows fluid flow in a direction from the cylinders to the reservoir. The pump assembly must be placed in the deactivated state to prevent spontaneous inflation. When in this state, the conical lip seal engages the cylindrical portion of the stem. If overpressure is generated, the reservoir poppet can be displaced towards the cylinder poppet. As this occurs, the increased fluid pressure levels force the conical lip seal to firmly abut the cylindrical portion of the stem, preventing increased pressure levels from reaching and displacing the cylinder poppet. Thus, spontaneous inflation is prevented.

In most of the embodiments, the force generated by an overpressurization of the reservoir is used to prevent fluid flow into the cylinders.

### BRIEF DESCRIPTION OF THE DRAWINGS

FIG. 1 Is a side-sectional view of a penile pump according to the teachings of the prior art.

FIG. 2 is a side-sectional view of a penile pump wherein the reservoir poppet has been attached to an outer wall of the reservoir chamber.

FIG. 3 is a side, partially sectional planar view of the attachment mechanism connecting the reservoir poppet to the outer wall of the fluid input chamber.

FIG. 4 is a side sectional view of housing for a penile pump having a tapered reservoir poppet and corresponding passageway, which plugs the fluid input during overpressure situation.

FIG. 5 is a side sectional view of housing for a penile pump having relief areas, which expand during an overpressure situation and seal against the reservoir poppet.

FIG. 6 is a side sectional view of the penile pump in FIG. 5, illustrated during an overpressure situation.

FIG. 7 is a side sectional view of the penile pump in FIG. 5, illustrated during a compression of the pump bulb.

FIG. 8 is a side sectional view of the penile pump in FIG. 5, illustrated during a reinflation of the pump bulb.

FIG. 9 is a side sectional view of the housing of a penile pump having relief areas which expand during an overpressure situation, and a termination chamber which cooperates with the cylinder poppet during the overpressure situation.

FIG. 10 is a side sectional view of a housing for a penile pump having a reservoir poppet coupled to the outer wall of the reservoir chamber via a connecting spring.

FIG. 11 is a side sectional view of the penile pump of FIG. 10 during an overpressurization situation.

FIG. 12 is a side sectional view of the penile pump of FIG. 10 when vacuum forces are generated by the pump bulb.

FIG. 13 is a side sectional view of the penile pump of FIG. 10 when both poppets have been manually opened.

FIG. 14 is a side sectional view of a housing for a penile pump wherein the reservoir poppet includes a relatively large biasing spring and a large diameter poppet face, which abuts the two-spaced lip seals.

FIG. 15 is a side sectional view of a housing for a penile pump having a reservoir poppet that includes a slidable valve seal that selectively includes a throughbore leading to an outlet in the reservoir poppet.

FIG. 16 is a side sectional view of the penile pump illustrated in FIG. 16 during a compression of the pump bulb.

FIG. 17 is a side sectional view of the penile pump illustrated in FIG. 16 when no forces are being generated.

FIG. 18 is a side sectional view of the penile pump illustrated in FIG. 16 when both poppets have been manually opened.

FIG. 18A is a perspective view of an alternate embodiment of a poppet usable in the penile pump in accordance with the present invention.

FIG. 19 is a side sectional view of a penile pump assembly including a conical lip seal and an annular ring that interact with a reservoir poppet having a grooved stem and an abutting face.

FIG. 20 is a side sectional view of the pump assembly of FIG. 19 while the pump bulb is reinflating after compression.

FIG. 20A is a side sectional view illustrating how the reservoir poppet may be spaced from the annulus to effect fluid flow.

FIG. 20B is front planar view of an annulus with a plurality of spacers.

FIG. 21 is a side sectional view of the pump assembly of FIG. 19 while the cylinders are being deflated.

FIG. 22 is a side sectional view of the pump assembly of FIG. 19 while in a deactivated state, which serves to inhibit spontaneous inflation.

### DETAILED DESCRIPTION OF THE INVENTION

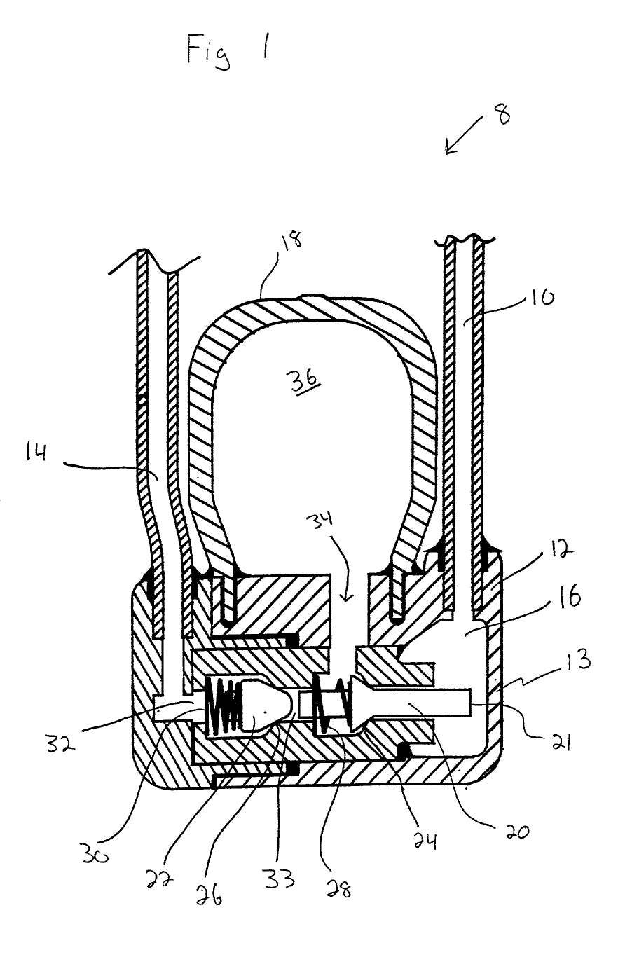

Referring to FIG. 1, a pump assembly is shown and generally referred to as 8. The pump assembly 8, as illustrated in FIG. 1, is essentially that of the prior art, but an understanding of the working elements of pump assembly 8, as illustrated in FIG. 1, is beneficial to understanding the operation of each embodiment of the present invention. Generally, the pump assembly 8 will be implanted into the patient's scrotum. A separate fluid-filled reservoir (not shown) is implanted in some other portion of the patient's body, usually in the abdomen. Fluidly connecting the reservoir to the pump assembly 8 is fluid input 10 which will usually be a flexible silicone tube. A pair of inflatable cylinders (not shown) are usually implanted in the patient's corpus cavernosae and are fluidly connected to pump assembly 8 via fluid output 14, which is also usually a flexible silicone tube.

In general, when pump assembly 8 is actuated, fluid is drawn from the reservoir through the pump assembly 8 and pumped into the cylinders. During the inflation process and until released by the patient, the pump assembly 8 maintains the fluid pressure in the cylinders, thus keeping them in their inflated state. When deflation is desired, the patient manipulates assembly 8, permitting fluid to transfer out of the inflatable cylinders and into the reservoir, thereby deflating the cylinders and returning them to a flaccid state.

Pump assembly 8 generally includes a housing 12 usually formed of silicone. Attached to housing 12 is a pump bulb 18, which includes a relatively large pump chamber 36. Fluid input 10 is coupled to the housing 12 and empties into a reservoir chamber 16. As such, fluid input 10 couples reservoir chamber 16 to the reservoir. A common passageway 33 is fluidly coupled to reservoir chamber 16 at one end of the housing 12, and is fluidly coupled to fluid output 14 at an opposite end of the housing 12. Similarly, the pump chamber 36 is fluidly coupled to the common passageway 33 via pump passageway 34.

Disposed within common passageway 33 is a reservoir poppet 20 which functions as a check valve. Reservoir poppet 20 is an elongated member having a contoured portion which abuts reservoir poppet valve seat 24 forming a fluid tight seal. A reservoir poppet spring 28 engages reservoir poppet 20 and biases reservoir poppet 20 against the reservoir poppet valve seat 24. Also disposed within common passageway 33 and in line with reservoir poppet 20 is cylinder poppet 22. Cylinder poppet 22 forms a second check valve within common passageway 33. Cylinder poppet 22 is biased by cylinder poppet spring 30 against cylinder poppet valve seat 26 in a normal state, thereby forming another fluid tight seal within common passageway 33. Reservoir poppet 20 is substantially longer than cylinder poppet 22. A front end of reservoir poppet 20 extends into reservoir chamber 16, in close proximity to an outer wall of housing 12. Furthermore, the front end of cylinder poppet 22 is in close proximity to the rear end of reservoir poppet 20. As such, the patient can manipulate both poppets 20 and 22 by compressing the wall of housing 12. Compression of the housing 12 will cause the reservoir poppet 20 to compress reservoir poppet spring 28 thus displacing the reservoir poppet 20 from reservoir poppet valve seat 24. This motion will also cause cylinder poppet 22 to be displaced from cylinder poppet valve seat 26 while compressing cylinder poppet spring 30. When both reservoir poppet 20 and cylinder poppet 22 are displaced from their respective valve seats, fluid is allowed to freely flow between reservoir chamber 16 and fluid output 14, and hence fluid is allowed to freely flow between the reservoir and the cylinders.

During a majority of the time, pump assembly 8 will be in the configuration shown in FIG. 1. That is, both reservoir poppet 20 and cylinder poppet 22 are abutting their respective valve seats 24 and 26, forming a fluid tight seal. When inflation is desired, pump bulb 18 is manually compressed by the patient. This forces the fluid in pump chamber 36 out through pump passageway 34 and into common passageway 33, under relatively high pressure. Because of the location of pump passageway 34 with respect to the reservoir poppet 20, this increased pressure causes reservoir poppet 20 to further abut reservoir poppet valve seat 24. This increased pressure is more than sufficient to remove cylinder poppet 22 from its abutment with cylinder poppet valve seat 26, by compressing cylinder poppet spring 30. As such, the pressurized fluid is allowed to pass through a portion of the common passageway 33 and into fluid output 14, where it eventually reaches an inflatable cylinder. When released, the pump bulb 18 expands back to its original configuration, creating negative pressure within pump chamber 36 and common passageway 33. This negative pressure draws cylinder poppet 22 towards valve seat 26 and simultaneously pulls reservoir poppet 20 away from valve seat 24. As such, fluid is drawn from the reservoir and into pump chamber 36 until the negative pressure is eliminated. Then, reservoir poppet spring 28 causes the reservoir poppet 20 to reseat itself against valve seat 24.

Repeated compression of pump bulb 18 eventually inflates the cylinders to a sufficient degree of rigidity for the patient. Once inflated, the fluid remaining in fluid output 14 is under a relatively high degree of pressure. This high pressure fluid aids cylinder poppet spring 30 in forcing cylinder poppet 22 against cylinder poppet valve seat 26 again forming a fluid tight seal and preventing fluid from within the cylinders from passing through (preventing deflation of the cylinders).

When the patient desires deflation of the cylinders, the wall of housing 13 is manually compressed. This compression forces reservoir poppet 20 away from reservoir poppet valve seat 24 and simultaneously causes cylinder poppet 22 to be removed from cylinder poppet valve seat 26. The pressurized fluid within the cylinders and fluid output 14 naturally returns to the reservoir via common passageway 33. Furthermore, the cylinders can be manually compressed forcing out any remaining fluid. Once the cylinders are satisfactorily emptied, the patient releases the grip on housing 12, thus allowing cylinder poppet 22 and reservoir poppet 20 to once again abut their respective valve seats 24 and 26.

As described above, pump assembly 8 (as shown in FIG. 1) works relatively well under normal circumstances. However, when the patient compresses the reservoir inadvertently through bodily movement, the pressure generated may be sufficient to remove reservoir poppet 20 and cylinder poppet 22 from their respective valve seats 24 and 26, thus spontaneously inflating the cylinders. When sufficient force is generated against the reservoir (or a similar component) to cause the fluid pressure to exceed the resistive characteristics of poppets 20 or 22, an overpressure situation has occurred. Of course, the only way to release this spontaneous inflation is to manually release the check valves.

To date, it has been very difficult to monitor and determine the pressures generated in an overpressure situation since each patient exhibits unique individual characteristics. Furthermore, each spontaneous inflation may result from a very different physical act on the part of the patient. However, it appears that pressure generated by compression of the reservoir results in a fluid pressure of up to 3 pounds per square inch (1.361 kg/25.4² mm) but may be as high as 6-8 pounds per square inch (2.722 kg/25.4² mm). Conversely, compression of the pump bulb 18 will usually generate pressures on the order of 20 pounds per square inch (9.072 kg/25.4² mm).

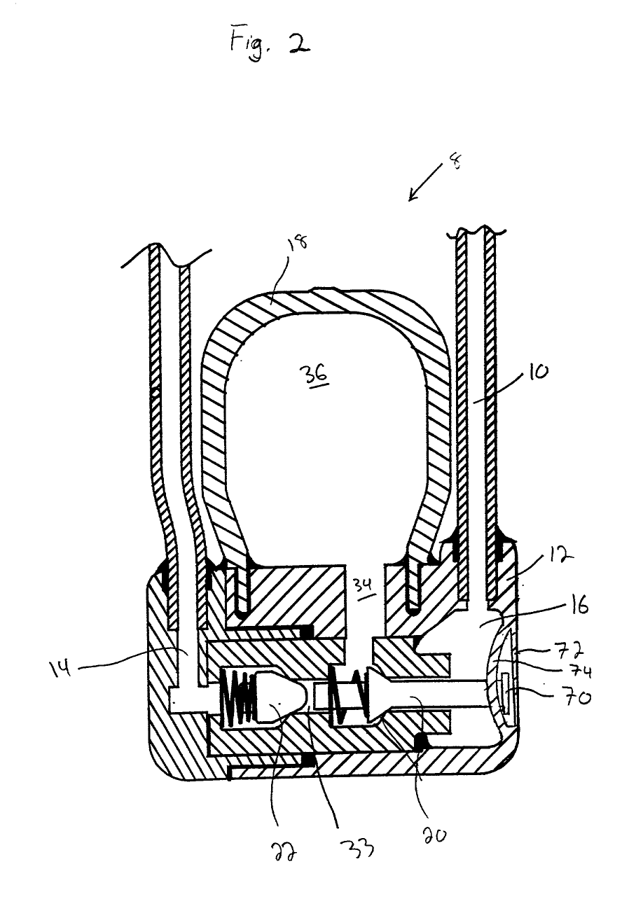

Referring to FIG. 2, a first embodiment of the present invention is illustrated. A fluid input 10 couples a reservoir to reservoir chamber 16. Reservoir poppet 20 has been modified to include a T-shaped tip 70. Tip 70 is secured to an outer reservoir chamber wall 72. Tip 70 is secured to the outer reservoir chamber wall by one or more connecting bands 74. Sufficient freedom of movement for reservoir poppet 20 is provided so that during normal operation reservoir poppet 20 can be dislodged from its abutment with reservoir poppet valve seat 24.

During an overpressure situation, the reservoir is compressed, pressurizing the fluid and directing it through fluid input 10 and into reservoir chamber 16. Outer reservoir chamber wall 72 has been made sufficiently flexible so that when this occurs, reservoir chamber 16 is caused to expand due to the increased pressure generated. As outer reservoir chamber wall 72 expands, connecting bands 74 coupled with tip 70 pull reservoir poppet 20 tightly against reservoir poppet seat 24. The overpressurization generated by the reservoir is used against itself to prevent fluid from reaching the cylinders and creating a spontaneous inflation.

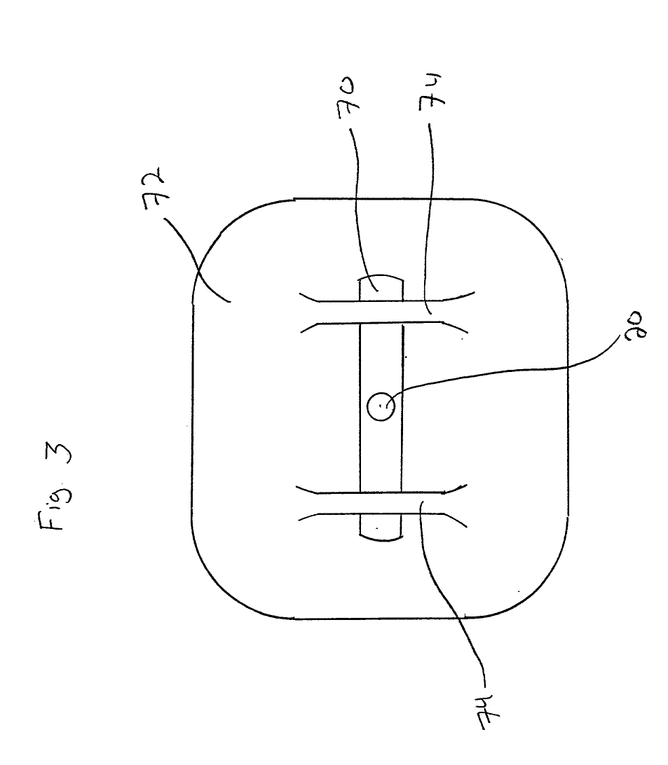

Referring to FIG. 3 a side partially sectional view is shown which helps illustrate the interior side of outer reservoir chamber wall 72. Tip 70 of reservoir poppet 20 is secured at each end by a connecting band 74 which overlaps tip 70 and is interconnected with outer reservoir chamber wall 72. Any interconnection of tip 70 or reservoir poppet 20 to outer reservoir chamber wall 72 is acceptable so long as during an overpressurization situation, reservoir poppet 20 is pulled against reservoir poppet valve seat 24 and during normal use sufficient flexibility is provided so that reservoir poppet 20 can be displaced from reservoir poppet valve seat 24 allowing the desired fluid flow.

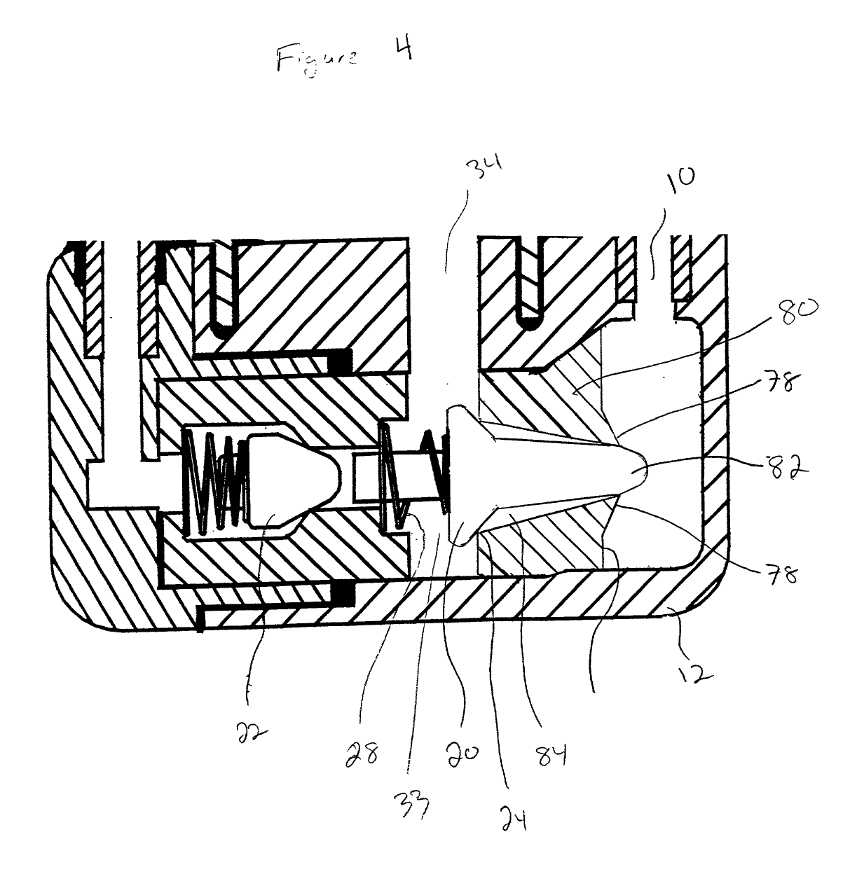

Referring to FIG. 4, a second embodiment of the present invention is illustrated. FIG. 4 illustrates the portion of housing 12 containing reservoir poppet 20 and cylinder poppet 22. Reservoir poppet 20 is an elongated member that terminates in a nose 82. A tapered reservoir passageway 84 is provided through a sidewall 80 located adjacent to fluid input 10. Located at the junction of the sidewall 80 and reservoir passageway 84 is a flap 78 that is able to flex, with respect to sidewall 80. Flap 78 is simply the terminus of sidewall 80 at the passageway 84, and will optimally be offset by some angle from the remainder of the sidewall 80.

As illustrated in FIG. 4, reservoir poppet 20 is in a sealed position. That is, fluid is not able to pass from fluid input 10 through tapered passageway 84 and beyond, because reservoir poppet 20 is sealed against sidewall 80 at reservoir poppet valve seat 24 and is held in place by spring 28. In addition, nose 82 of reservoir poppet 20 contacts flap 78, providing a further seal. The remainder of passageway 84 is open between reservoir poppet 20 and sidewall 80.

In normal use, reservoir poppet 20 is pulled away from its sealed position by a vacuum created at pump passageway 34. This allows fluid to pass from fluid input 10, through passageway 84, and then through common passageway 33 into pump bulb 18. During a compression of pump bulb 18, reservoir poppet 20 is further pressed against valve seat 24.

During an overpressure situation, the fluid pressure in the reservoir and hence within fluid input 10 will increase. This increased pressure is applied evenly within fluid input 10, however flaps 78 are able to give in response to these forces. As such, flap 78 will be forced against a portion of reservoir poppet 20. The shape of reservoir poppet 20 and passageway 84 are chosen so that as flap 78 is pressed against reservoir poppet 20, a strong seal is formed. In other words, sufficient give is provided in sidewall 80, particularly at and behind flap 78 (due to its shape and flexibility) so that increased pressure causes a fluid tight encasement of poppet 20 rather than a displacement of poppet 20. Therefore, reservoir poppet 20 remains sealed and spontaneous inflation is prevented. While one specific configuration of this concept is shown in FIG. 4, it is to be understood that a wide variety and combinations of the disclosed teachings may be used while achieving the same result. The shape of the reservoir poppet 20, passageway 84, and the location and shape of flap 78 are extremely variable so long as these elements work together to form a fluid tight seal during an overpressure situation.

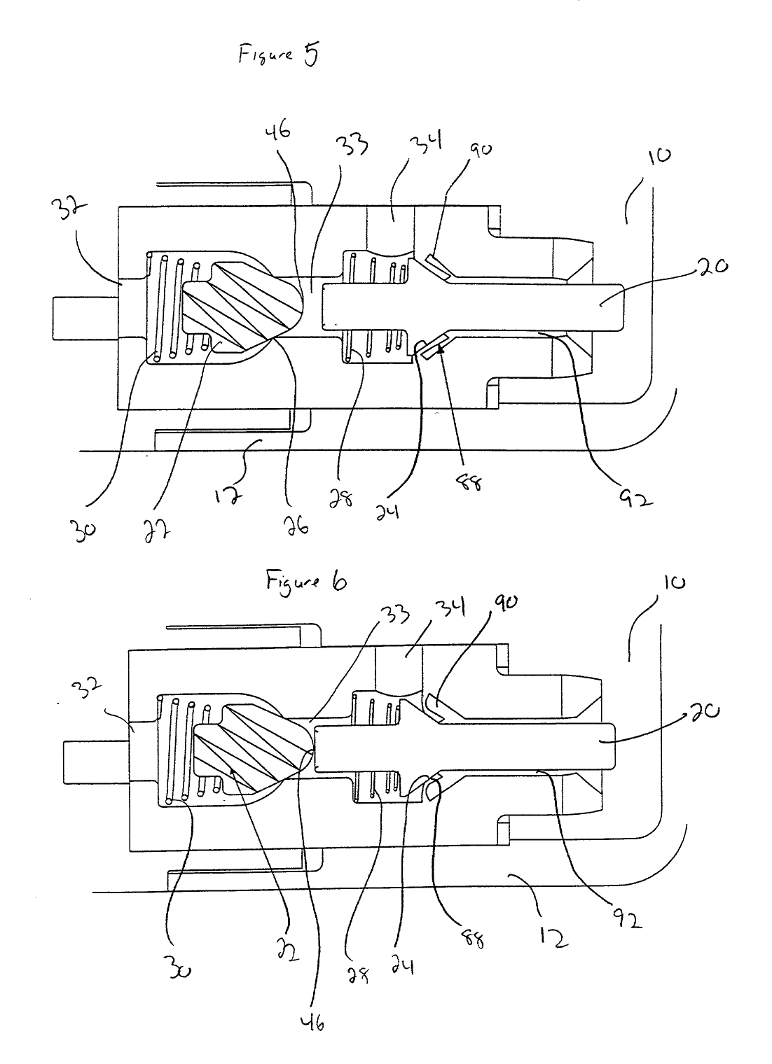

Referring to FIG. 5, a third embodiment is illustrated. Reservoir poppet 20 is an elongated member that extends from common passageway 33, through poppet passageway 92 and into fluid input 10. As with many of the above embodiments, in one position the reservoir poppet 20 abuts reservoir poppet valve seat 24. Similarly, reservoir poppet 20 is only expected to be removed from valve seat 24 during a re-expansion of a compressed pump bulb 18. To prevent the removal of the reservoir poppet from valve seat 24 during an overpressure situation, relief area 90 has been formed within the housing 12. Formation of relief area 90 creates a flexible valve 88. Flexible valve 88 forms a part of the reservoir poppet valve seat 24, and appears as shown in FIG. 5, under normal circumstances.

FIG. 6 illustrates an overpressure situation where the pressure of the fluid in fluid input 10 and poppet passageway 92 is relatively high. Rather than forcing reservoir poppet 20 from valve seat 24, this overpressure causes relief area 90 to expand; which in turn causes flexible valve 88 to even more firmly abut reservoir poppet 20. Depending upon the particular arrangement chosen, such an expansion of relief area 90 may cause some compression of reservoir poppet spring 28. In other words, reservoir poppet 20 is caused to move towards the cylinder poppet 22. Such motion will normally allow a spontaneous inflation to occur. However, in this embodiment, it is the movement of valve seat 24 that moves reservoir poppet 20, as such, a fluid seal is not only maintained, it is made stronger. To further support reservoir poppet 20, nose 46 of cylinder poppet is located in close proximity to the rear of reservoir poppet 20. As such, when expansion of relief area 90 causes a small amount of movement of reservoir poppet 20, reservoir poppet 20 is caused to abut cylinder poppet 22. Therefore, any further movement of reservoir poppet 20 requires compression of both reservoir poppet spring 28 and cylinder poppet spring 30. This combination of spring forces provides a relatively high resistive force opposing further movement of reservoir poppet 20, even during an overpressure situation. This combined with the expandable characteristics of relief area 90 prevents a spontaneous inflation from occurring. Of course, the relief area 90 can be fashioned to prevent such spontaneous inflation without causing the reservoir poppet 20 to engage cylinder poppet 22.

FIG. 7 illustrates a state where pump bulb 18 is being compressed, forcing fluid around cylinder poppet 22 and out through cylinder poppet output 32. Simultaneously, reservoir poppet 20 is forced towards fluid input 10, causing flexible valve 88 to collapse against the inner portions of relief area 90. Once again, the strength of the seal at valve seat 24 is increased during such movement.

Immediately after the state shown in FIG. 7 occurs, pump bulb 18 is released. As illustrated in FIG. 8, this creates a vacuum which pulls cylinder poppet 22 against cylinder poppet valve seat 26 and pulls reservoir poppet 20 away from valve seat 24; thus allowing fluid from the reservoir to flow into pump bulb 18. Flexible valve 88 is created with sufficient rigidity to resist being forced against reservoir poppet 20 while fluid is flowing through poppet passageway 92 and into pump bulb 18. Furthermore, the previous compression of flexible valve 88 against poppet 20 (FIG. 7) substantially evacuates relief area 90. Therefore when reservoir poppet 20 is initially pulled from valve seat 24, relief area 90 will remain in an evacuated state while fluid flow begins. The system is configured so that relief area 90 will not totally fill (and expand) with fluid and seal against reservoir poppet 20 until pump bulb 18 has been refilled. This can be done by making flexible valve 88 too rigid to allow such a seal to be formed in this state; providing for a sufficient amount of reservoir poppet 20 movement to prevent the flexible valve 88 from reaching poppet 20, even when relief area 90 is completely expanded; or simply imparting sufficient rigidity in flexible valve 88 so that the time is takes to expand relief area 90 is greater than the time it takes to refill pump bulb 18.

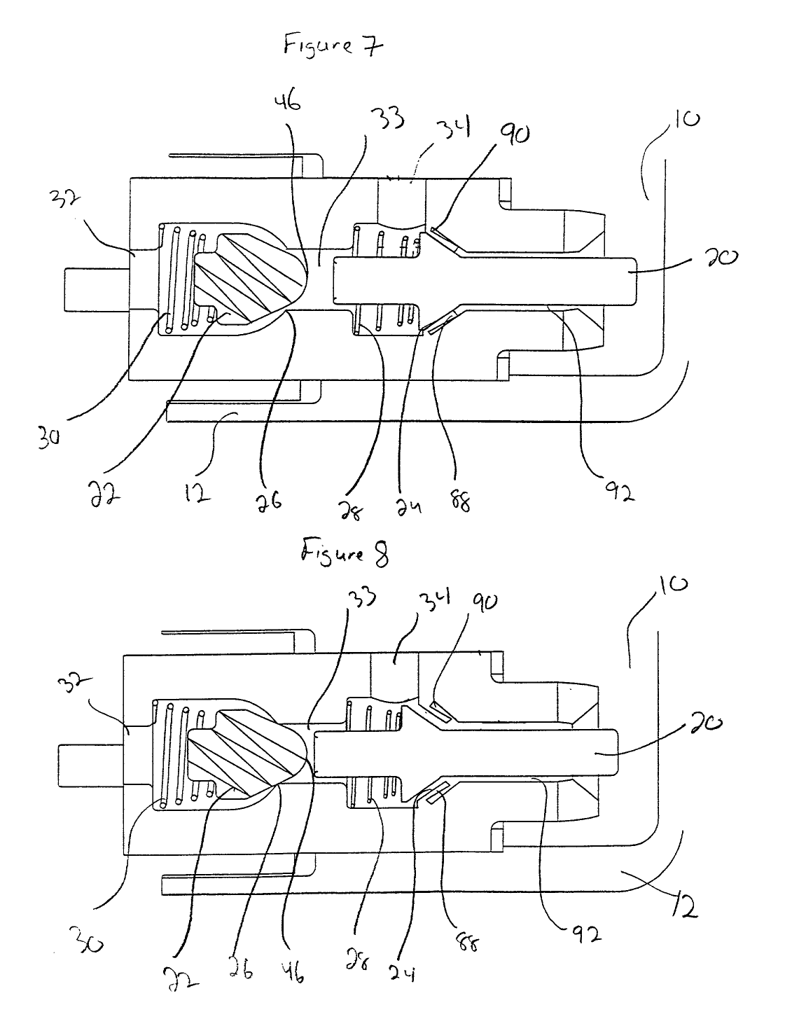

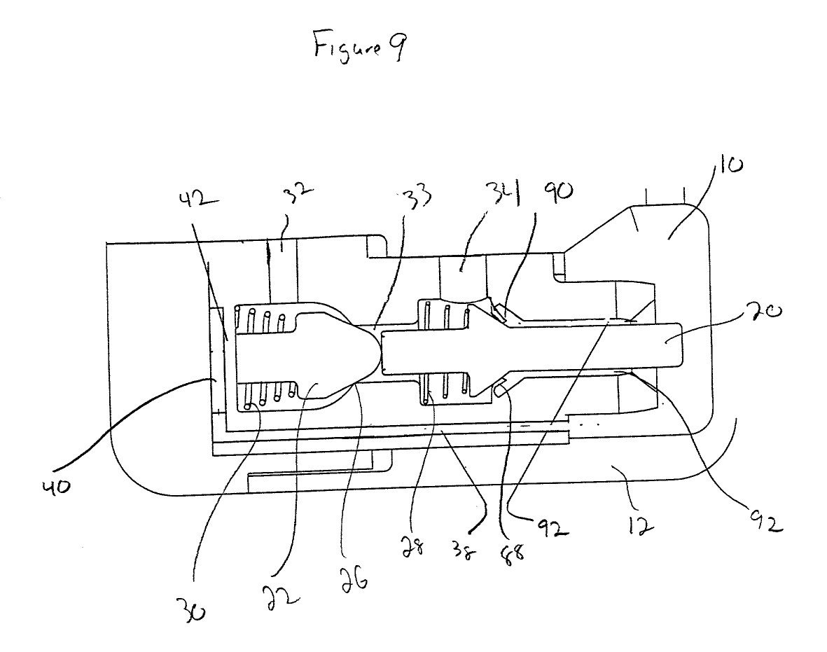

FIG. 9 illustrates a fourth embodiment utilizing a combined solution to avoid spontaneous inflation. Namely, relief area 90 has been provided and works as described above. In addition, bypass passageway 38 has been provided which fluidly connects fluid input 10 to termination chamber 40. Termination chamber 40 includes abutting wall 42, which acts as a diaphragm when an overpressure situation occurs. These two mechanisms will act in concert to prevent a spontaneous inflation from occurring. One advantage of this arrangement is that nose 46 of the cylinder poppet 22 will be displaced towards the rear of reservoir poppet 20 via an expansion of termination chamber 40. This force opposes the movement of the reservoir poppet 20, in the opposite direction that is generated from an expansion of relief area 90. In essence, the force generated by the overpressure is caused to directly oppose itself, which in turn prevents spontaneous inflation.

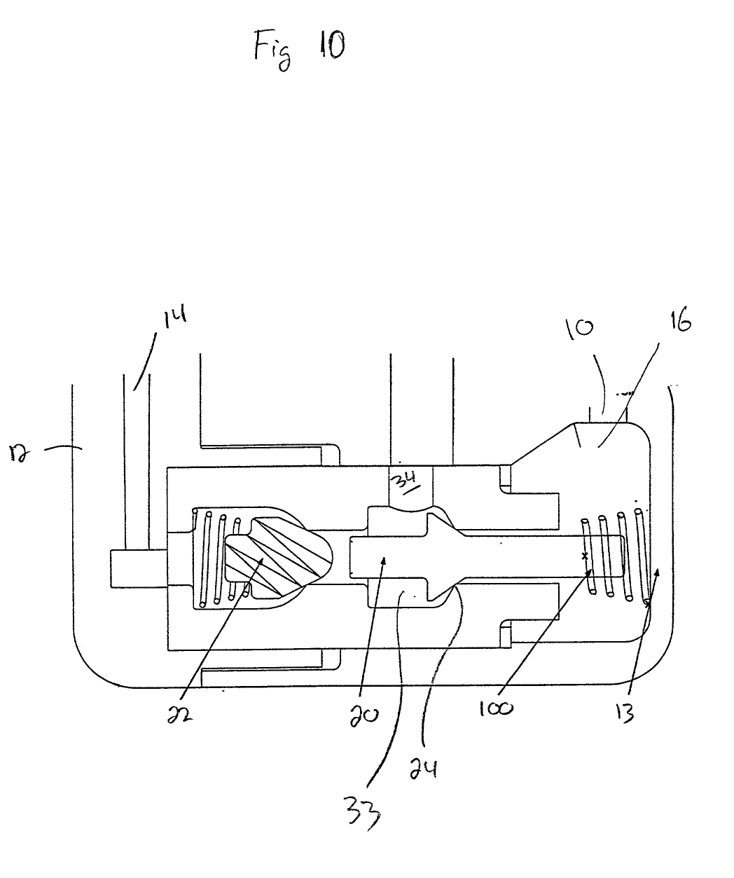

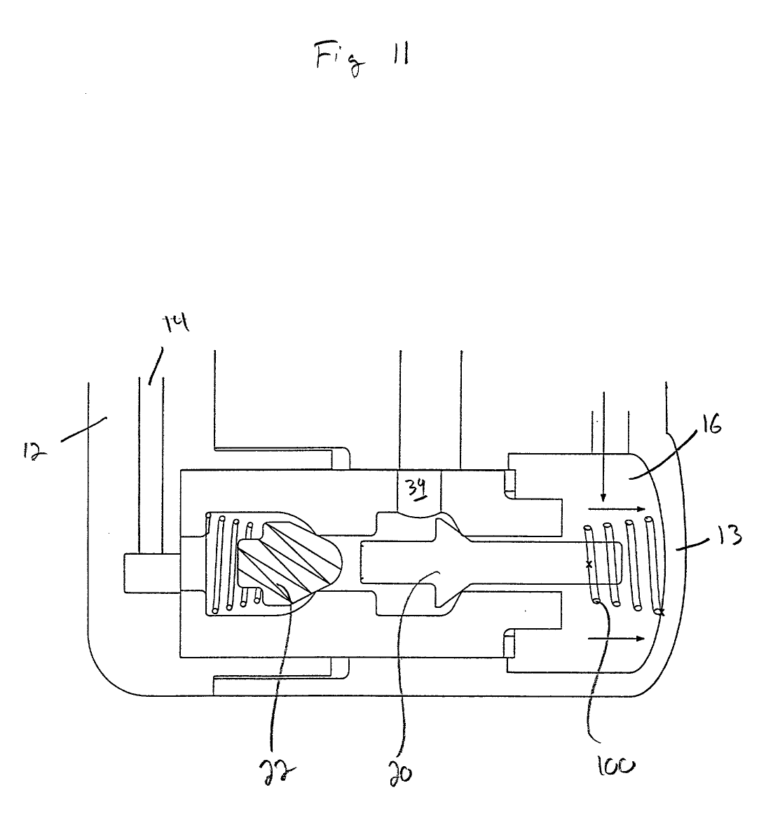

Referring to FIG. 10, a fifth embodiment to the present invention is illustrated. Housing 12 includes a fluid input 10 that is in fluid communication with fluid output 14 through a reservoir chamber 16 and a common passageway 33. Common passageway 33 is selectively occluded by a reservoir poppet 20 and cylinder poppet 22 which are both biased towards a closed position. A portion of reservoir poppet 20 is physically connected to a connection spring 100. The opposite end of connection spring 100 is attached to a wall 13 of housing 12. Connections to spring 100 are biased to maintain the configuration illustrated in FIG. 10.

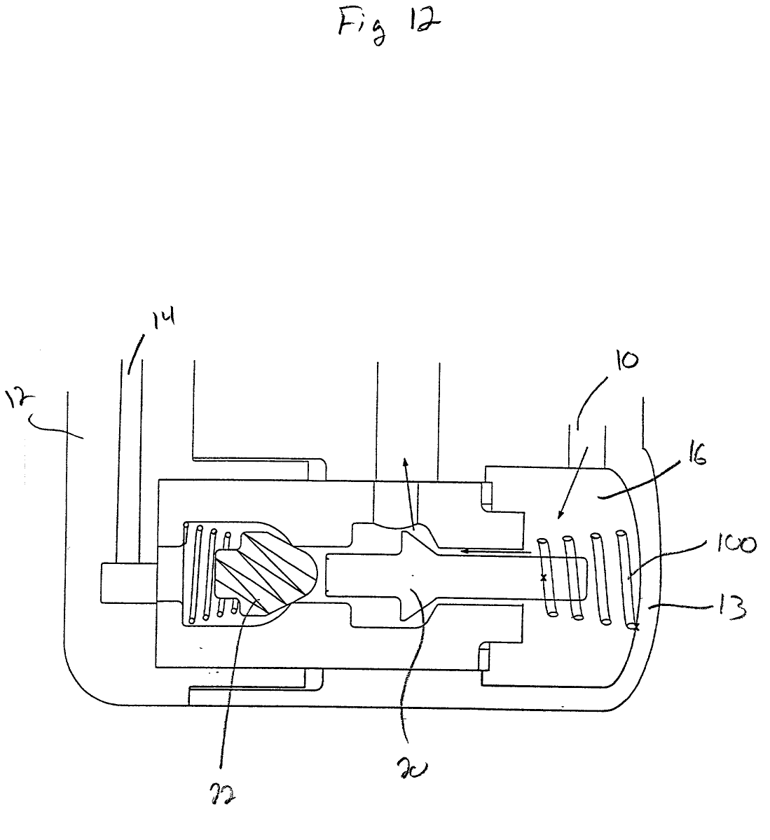

FIG. 11 illustrates what occurs during an overpressurization situation. As increased fluid pressure is generated, wall 13 in reservoir chamber 16 is caused to expand outward as indicated by the arrows. Since connection spring 100 is fixedly attached to wall 13, the tension generated by expanding spring 100 serves to pull reservoir poppet 20 firmly against valve seal 24, creating an even more fluid tight seal.

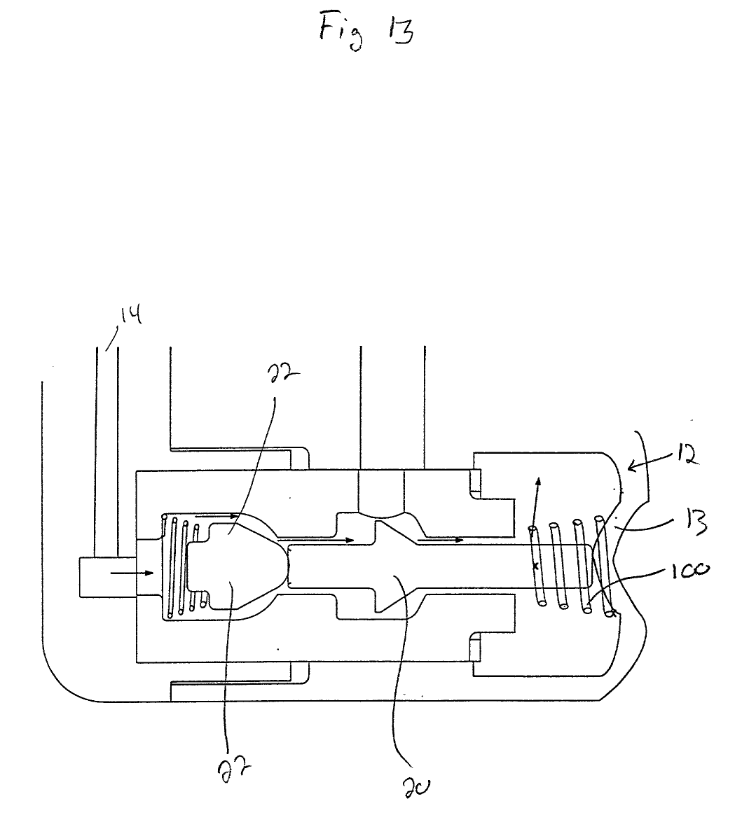

Once pump bulb 18 has been compressed and released, vacuum forces are generated which unseat reservoir poppet 20. This situation is illustrated in FIG. 12. Thus, despite an overpressurization situation wherein wall 13 is expanded outwardly and connection spring 100 is pulling against reservoir poppet 20, the vacuum forces generated, are sufficient to unseat reservoir poppet 20 and allow fluid flow into pump bulb 18 (as shown by flow arrows A).

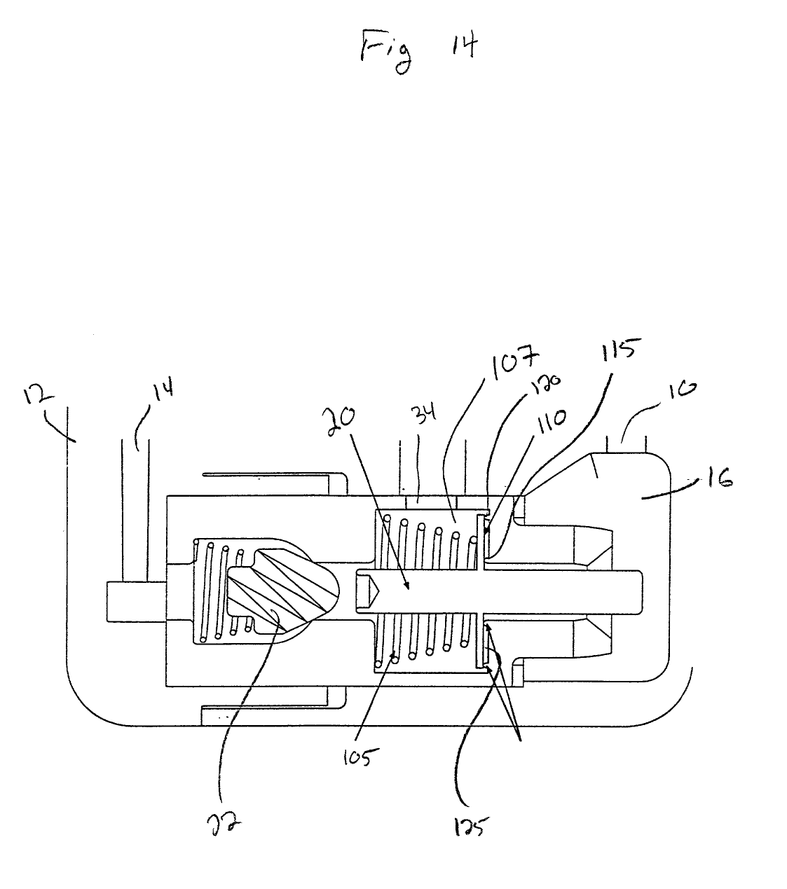

When so desired, wall 13 is compressed causing reservoir poppet 20 to unseat itself and contact cylinder poppet 22 which, in turn, unseats that valve as well. Thus, fluid from the cylinders can be returned to the reservoir. This situation is illustrated in FIG. 13 and illustrates how the interaction of connection spring 100 and reservoir poppet 20 will facilitate this movement.

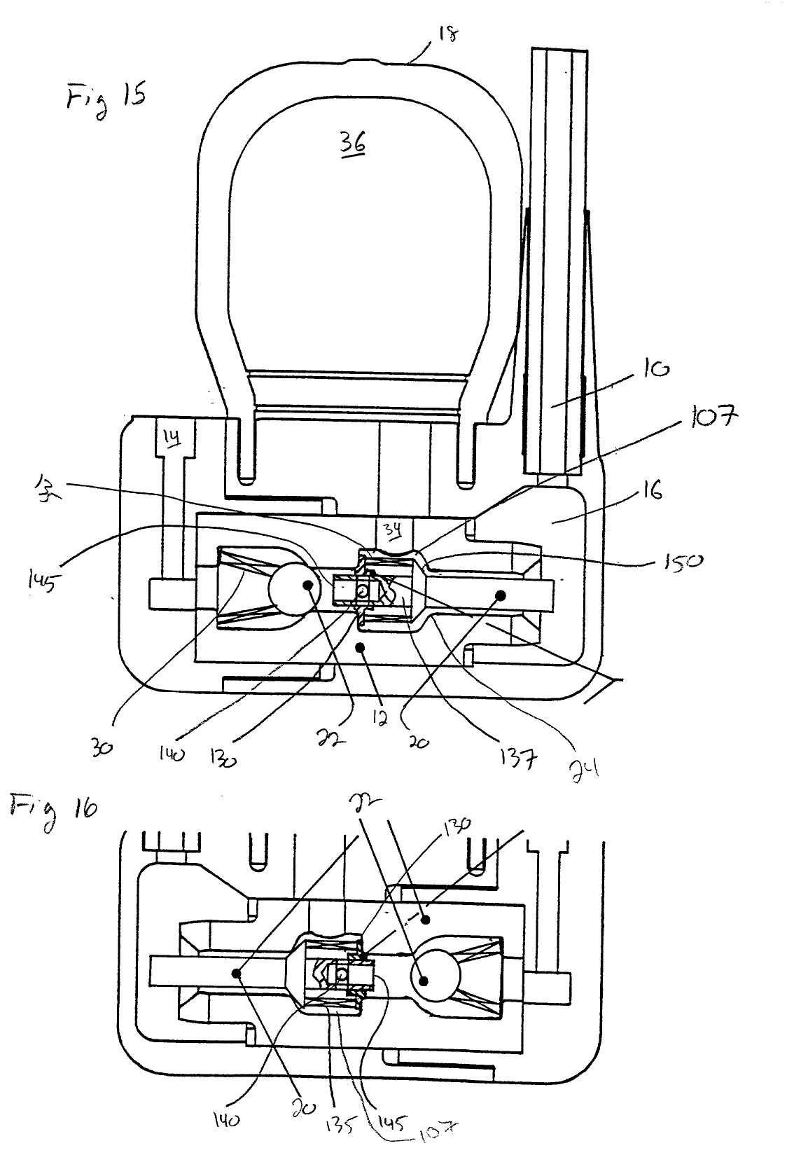

Referring to FIG. 14, a sixth embodiment of the present invention is illustrated. A biasing spring 105, exerting a large amount of force, is coupled to reservoir poppet 20 keeping it in its closed position. Because of the large amount of force being exerted, biasing spring 105 will be able to resist high forces generated during an overpressurization situation and, thus, preventing spontaneous inflation.

Because biasing spring 105 is significantly stronger than those in the previous embodiments, it also makes it harder to open reservoir poppet 20 with the level of vacuum forces generated by the pump bulb 18. To overcome this issue, poppet face 110 is made significantly larger than in the previous embodiments. That is, the surface area of poppet face 110 has a diameter that approximates the diameter of intermediate chamber 107, which houses reservoir poppet 20. Though the amount of pressure generated by the suction of release pump bulb 18 will be fixed, by increasing the surface area of poppet face 110, the negative force generated will be greatly increased and will allow biasing spring 105 to be overcome.

As illustrated, the portion of housing 12 in contact with poppet face 110 when reservoir poppet 20 is closed, is not simply a planar configuration. As a practical matter, it is too difficult to manufacture a planar surface which will flushly and repeatedly coact with a planar poppet face 110 to consistently form a fluid-tight seal. Instead, a pair of flexible lip seals is provided. That is, inner lip seal 115 and outer lip seal 120 are provided and define a recessed portion 125 between them. Outer lip seal 120 contacts an outer portion of poppet face 110 preventing suction forces from interacting with the rear portion of poppet face 110 and holding it in place during a refilling of pump bulb 18. Inner lip seal 115 prevents fluid pressure generated during an overpressurization situation from acting against a majority of poppet face 110, which would otherwise eliminate much of the benefit of having a larger biasing spring 105. Lip seal 115 acting in conjunction with the forces generated by biasing spring 105 allows poppet face 110 to form a fluid-tight seal despite any irregularities in either poppet face 110 or housing 112. During an overpressurization situation, pressurized fluid from reservoir chamber 16 interacts with only a very small area of poppet face 110. The force generated will be insufficient to move biasing spring 105, thus, reservoir poppet 20 will remain in the sealed position preventing spontaneous inflation.

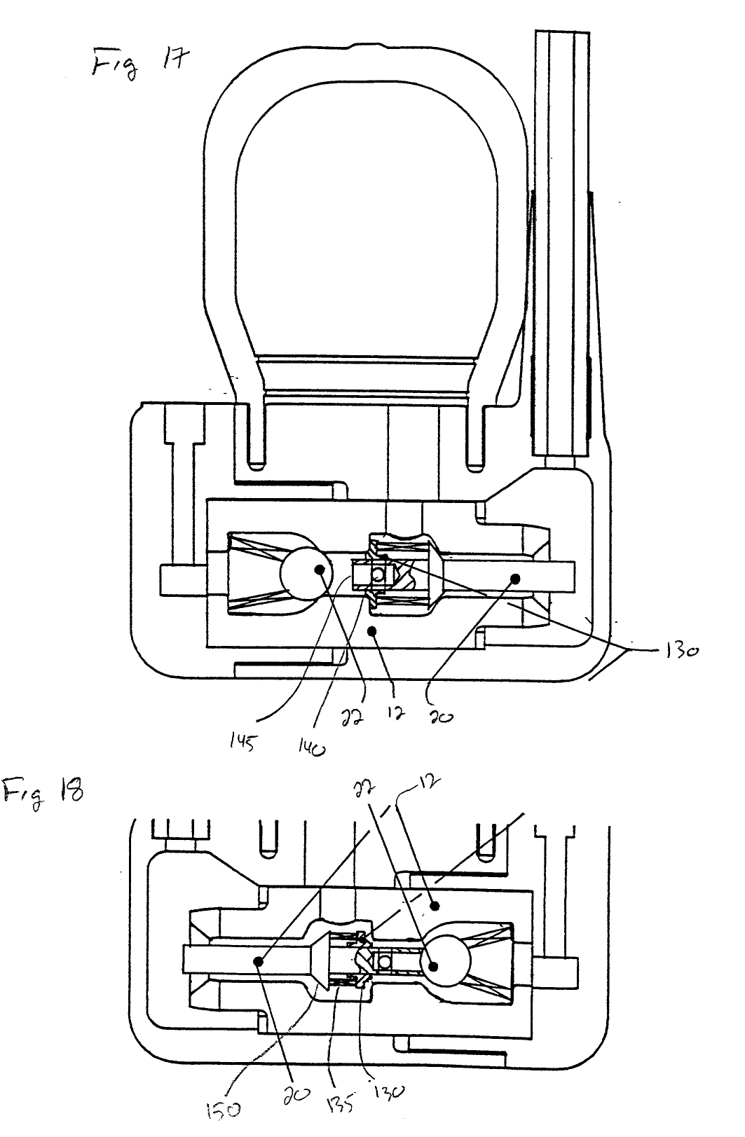

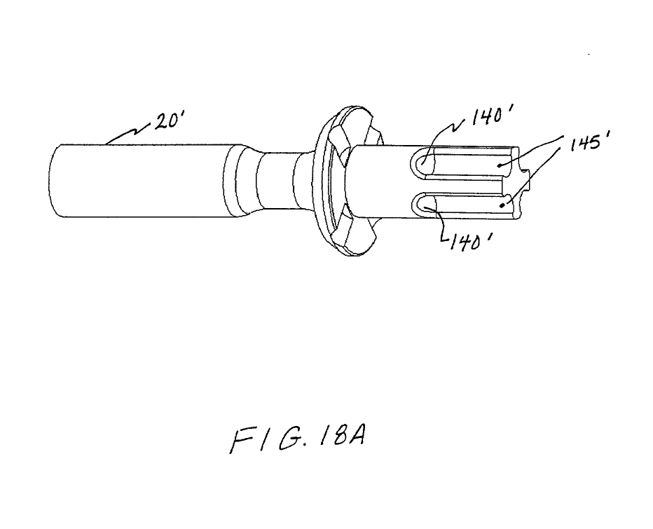

Referring to FIG. 15, a seventh embodiment of the present invention is illustrated. Once again, a reservoir poppet 20 and cylinder poppet 22 are provided to selectively occlude a common passageway 33 between a reservoir chamber 16 and a fluid output 14. As in the previous embodiments, a front face 150 of reservoir poppet 20 abuts valve seal 24 to prevent fluid flow from reservoir chamber 16. In this embodiment this occurs in two different situations. That is during a compression of pump bulb 18 (as illustrated in FIG. 16) and during an unused situation when no overpressurization is occurring (as illustrated in FIG. 17).

Extending behind front face 150 is a rear section 137 of poppet 20. At least a portion of rear section 137 is hollow and is in fluid communication with throughbore 140 (a plurality of throughbores 140 can also be provided). Outlet 145 forms a terminus of rear section 137 and is also in fluid communication with the hollowed out portion. A valve sleeve 130 slides over rear section 137 and is held in a spaced relationship from front face 150 by slide spring 135 which biases front face 150 away from valve sleeve 130. The movement of valve sleeve 130 with respect to rear section 137 selectively seals and unseals throughbore 140.

As illustrated in FIG. 17, under normal conditions valve sleeve 130 is abutting a portion of housing 12. Slide spring 135 biases front face 150 of poppet 20 against valve seal 24. In this situation, it is front face 150 that prevents fluid flow from reservoir 16.

During an overpressurization situation, as illustrated in FIG. 16, the forces generated within reservoir chamber 16 serve to unseat front face 150 causing it to move away from valve seat 24. To accomplish this, slide spring 135 must be at least partially compressed. In other words, overpressurization forces must be sufficient to compress slide spring 135 to cause this to occur. As front face 150 is unseated, rear section 137 moves through valve sleeve 130, since valve sleeve 130 is pressed firmly against a portion of housing 12. This action causes throughbore 140 to be occluded by valve sleeve 130. Therefore, even though pressurized fluid is able to enter into chamber 107, it is unable to pass through valve sleeve 130 and enter throughbore 140. Consequently, pressurized fluid never reaches cylinder poppet 22 and is, therefore, unable to unseat it and cause spontaneous inflation.

During compression of the pump bulb 18 (FIG. 16), pressurized fluid enters intermediate chamber 107 forcing front face 150 to firmly abut against valve seal 24. At the same time valve sleeve 130 is pressed firmly against its respective portion of housing 12. Since valve sleeve and front face 150 are spaced at their maximum distance, throughbore 140 is exposed and pressurized fluid from pump bulb 18 is able to pass through and unseat cylinder poppet 22 leading to an inflation of the cylinders.

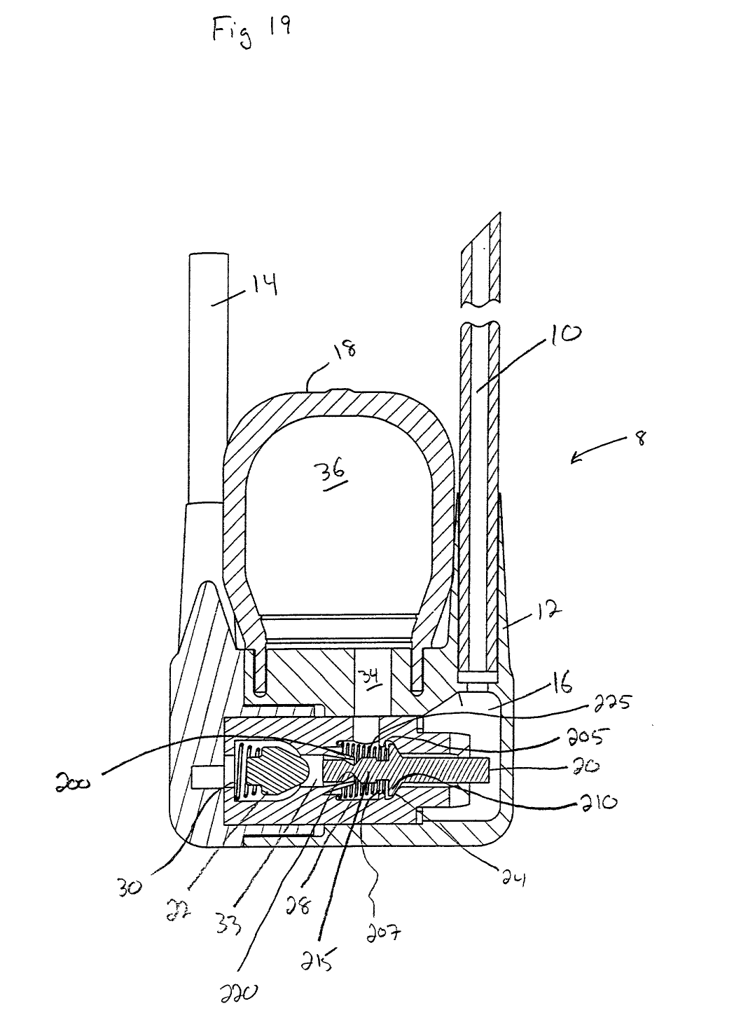

FIG. 18 illustrates how a manual release of a reservoir poppet 20 can unseat both the reservoir poppet 20 and cylinder poppet 22 allowing for deflation of the cylinders. Sleeve 130 is forced toward front face 150 by the pressure in the cylinders once cylinder poppet 20 is unseated.

Referring to FIG. 18A, a poppet 20′ is disclosed that can alternatively be incorporated into previous embodiments of the invention in place of poppet 20. The alternative poppet 20′ includes a plurality of flutes 145′ that loosely correspond in function to the output 145 discussed previously. Similarly, the lower, curved ends 140′ of the flutes 145′ loosely correspond in function to the throughbore 140 discussed previously.

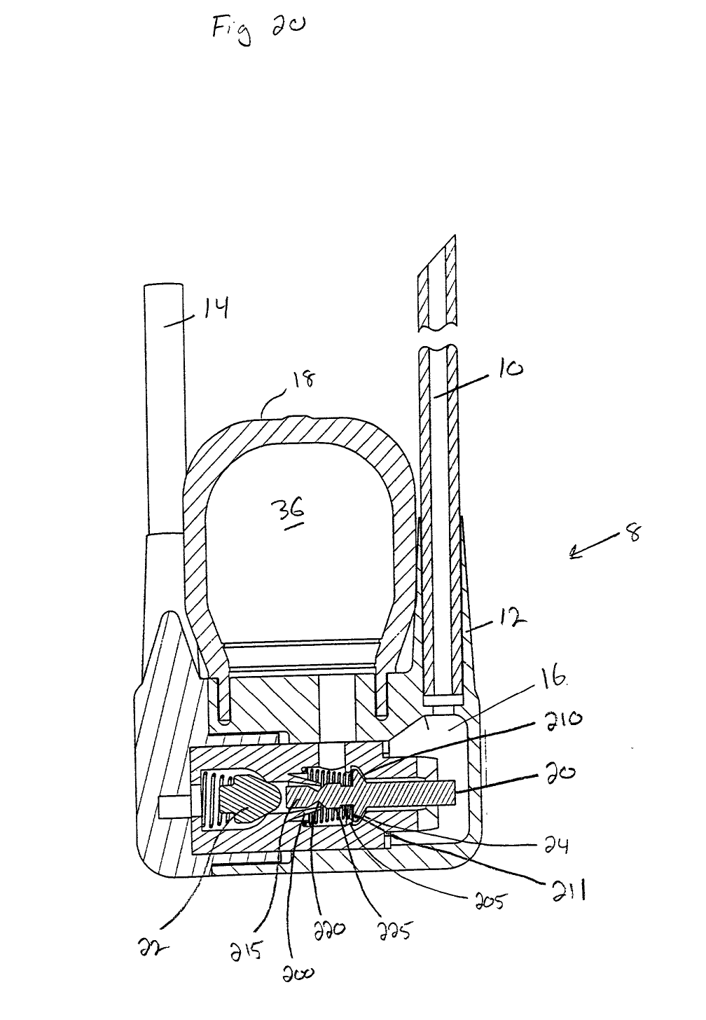

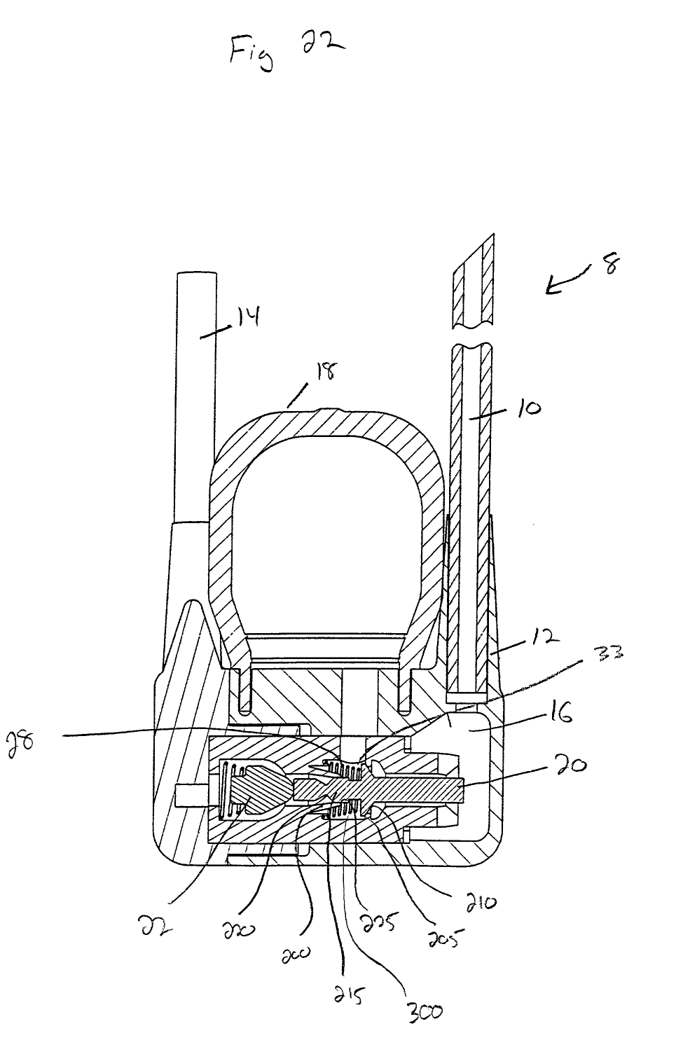

Referring to FIGS. 19-22, an eighth embodiment of the present invention is illustrated. Housing 12 includes common passageway 33 that fluidly couples reservoir chamber 16 to fluid output 14 and is fluidly coupled to pump passageway 34. Housing 12 also includes a tapered reservoir poppet valve seat 24 configured to interact with a similarly tapered front face 210 of reservoir poppet 20. An annulus 205 is formed within housing 12 and is spaced away from, but proximate to, valve seat 24. Annulus 205 is configured to provide an opening 207 that is slightly smaller than front face 210. Annulus 205 is a semi-rigid portion of housing 12 that allows passage of front face 210 through opening 207 by moderate deflection. In other words, even though front face 210 is slightly larger than opening 207, it can still be forced therethrough. (This relationship is more clearly seen by comparing FIG. 19 with FIG. 21.)

Housing 12 also includes a conical lip seal 200, which is positioned just forward of cylinder poppet 22. Conical lip seal 200 is a flexible member that interacts with a stem 215 of reservoir poppet 20. Stem 215 is generally cylindrical and includes a V-shaped groove 220 extending around its circumference. Groove 220 thus defines a medial stem section 225 that lies between groove 220 and front face 210. Medial stem section 225 is generally cylindrical.

Reservoir poppet 20 can be placed into three distinct configurations that define an activated state, a deactivated state, and a draining state. In the activated state, pump bulb 18 can be used to inflate the cylinders. Reservoir poppet 20 is also maintained in the activated state while the cylinders are to remain inflated. In the draining state illustrated in FIG. 21, the cylinders can be emptied. Reservoir poppet 20 is placed in the deactivated state during periods of non-use to prevent spontaneous inflation.

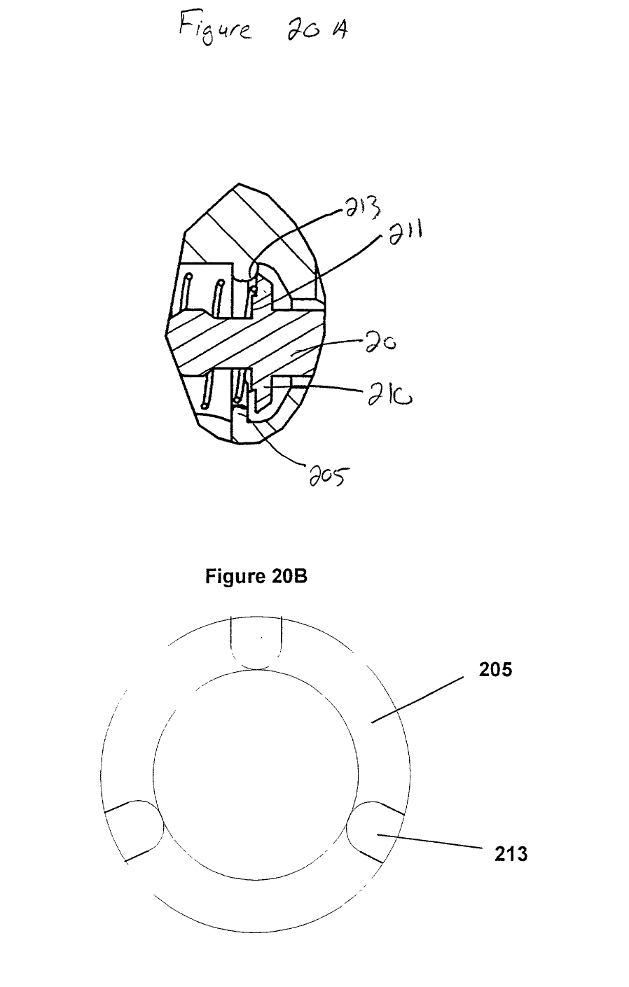

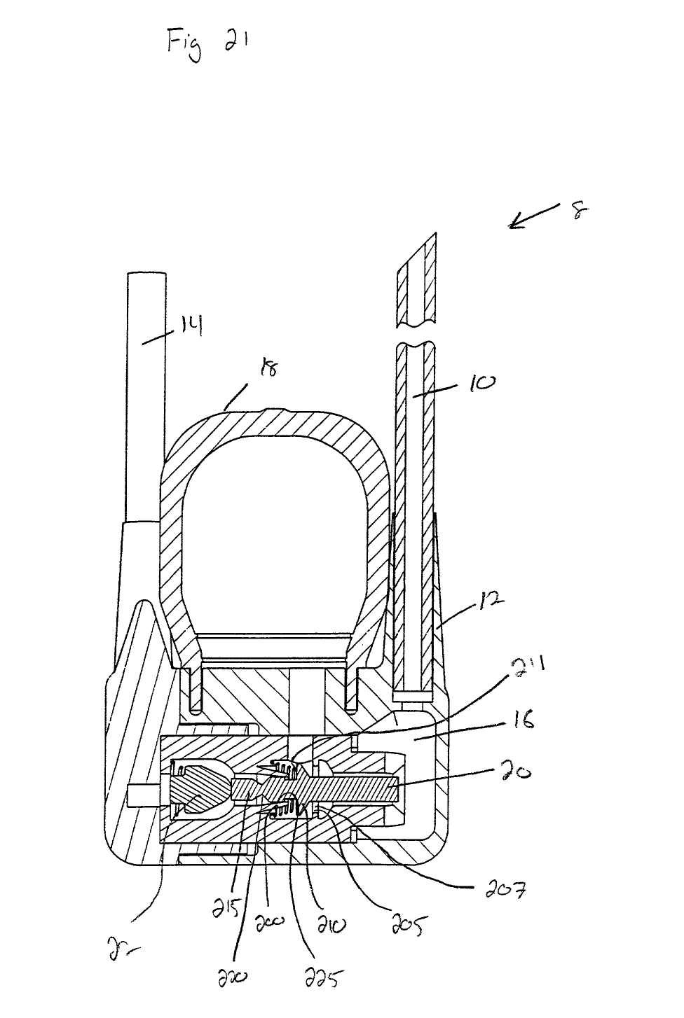

FIGS. 19 and 20 illustrate pump assembly 8 in the activated state. Front face 210 is positioned between annulus 205 and valve seat 24. When so positioned, reservoir poppet spring 28 biases front face 210 against valve seat 24. If pump bulb 18 is compressed, the fluid pressure generated reinforces the biasing action of reservoir poppet 28, and causes front face 210 to further abut valve seat 24. At the same time, cylinder poppet 22 is unseated and fluid is forced into the cylinders. When reservoir poppet 20 is so positioned, V-shaped groove 220 is aligned with conical lip seal 200. This effectively prevents conical lip seal 200 from interfering with fluid flow in either direction. That is, the configuration of conical lip seal 200 is such that it cannot effectively prevent fluid flow in a direction from cylinder poppet 22 towards reservoir chamber 16. Fluid flow in the opposite direction is also unhindered (in the activated state) because groove 220 permits fluid pressure levels to increase “underneath” conical lip seal 200 (i.e., between lip seal 200 and stem 215), thus fluid flow is permitted from pump chamber 36 to the cylinders. FIG. 19 illustrates this configuration during a compression of pump bulb 18.

FIG. 20 illustrates the configuration of the components during a release of pump bulb 18. The vacuum generated works with the biasing force of cylinder poppet spring 30 to cause cylinder poppet 22 to seal. The vacuum forces also cause front face 210 to be pulled away from valve seat 24. This allows fluid to flow from reservoir chamber 16 into pump chamber 36. While the vacuum forces are sufficient to unseat front face 210, they are insufficient to cause it to pass through annulus 205; thus, back face 211 of reservoir poppet 20 abuts annulus 205 or (depending on the spring forces involved) is held between annulus 205 and valve seat 24. In either case, fluid as able to flow into pump chamber 36. After a number of compressions of pump bulb 18, the cylinder will be inflated. While the cylinders are to remain inflated, pump assembly 8 is kept in the activated state.

During a release of pump bulb 18, the vacuum forces generated may be sufficient to cause back face 211 to seal against annulus 205. If this occurs, the pump assembly may lock up and remain in this position. That is, pump bulb 18 will be at least partially compressed and the vacuum generated will be sufficient to keep reservoir poppet 20 sealed against annulus 205, preventing fluid from moving from the reservoir to pump chamber 36. All that need be done to relieve the vacuum is manually compress the sidewall to cause reservoir poppet 20 to unseat.

This situation may be confusing to patients and they may not realize the nature of the problem. Thus, a modified annulus 205 (and/or a variation in reservoir poppet 20) can be provided to prevent the situation from occurring. Referring to FIGS. 20A and 20B, such a modified annulus 205 is illustrated. Annulus 205 includes a number of spacers 213 positioned about annulus 205 and facing valve seat 24. Spacers 213 are positioned so that when rear face 211 is in contact with them, there is still a fluid path around reservoir poppet 20 and through annulus 205. That is, there is never an opportunity for rear face 211 to seal against annulus 205.

The nature and number of spacers 213 can vary. Providing three spacers allows full support of rear face 211. That is, rear face 211 is not caused to pivot by only being supported at one or two points. This pivoting action is not necessarily detrimental, and one or two spacers 213 could be utilized. More could also be utilized, so long as sufficient fluid flow is permitted. The actual size and shape of spacers 213 will depend upon the methods utilized to form them. Any size, shape and configuration is permissible so long as fluid flow sufficient to prevent the above described vacuum lock is permitted. Finally, spacers 13 could be attached to rear face 211 rather than annulus 205 to permit appropriate fluid flow.

Alternatively, various other methods could be employed to achieve the same result. So long as fluid flow around rear face 211 and through annulus 205 is permitted, this potential problem is avoided. There are solutions other than providing spacers. For example, one or more grooves could be cut into rear face 211 to achieve the same result. Various other access ports or passageways could likewise be provided. Of course, these various techniques could be combined in any number of ways.

After use, when the operator wishes to deflate the cylinders, the sidewalls of housing 12 are compressed. This forces reservoir poppet 20 to move from the activated position, past the deactivated position (as shown in FIG. 22) and into the draining state, by causing front face 210 to move through annulus 205 to the position illustrated in FIG. 21. Furthermore, this movement of reservoir poppet 20 causes it to engage cylinder poppet 22 and unseat it as well as moving front face 210 away from annulus 205. Fluid is then able to flow from the cylinders into the reservoir.

When the cylinders are satisfactorily deflated, housing 12 is released. Referring to FIG. 22, reservoir poppet spring 28 biases front face 210 against annulus 205. As shown, reservoir poppet 20 is in the deactivated position. In this position, conical lip seal 200 engages medial stem section 225, which is cylindrical in nature and approximates conical lip seal 200 in size and shape. Should a compression of the reservoir cause an overpressure situation, increased fluid pressure will force reservoir poppet 20 to be moved back from annulus 205 and allow reservoir pressure to enter intermediate space 300. Without lip seal 200, reservoir pressure would enter common passageway 33 and open cylinder poppet 22 causing spontaneous inflation. However, reservoir pressure will act on conical lip seal 200 causing it to firmly seal against medial stem section 225, thus preventing fluid pressure from acting on cylinder poppet 22 and thus preventing spontaneous inflation.

The operator must place pump assembly 8 in the deactivated state during periods of non-use to effectively prevent spontaneous inflation. When the operator desires to inflate the cylinders and pump assembly 8 is in the deactivated state, all that is required is a compression of pump bulb 18. As pump bulb 18 is compressed, fluid pressure levels within intermediate space 300 are rapidly increased to relatively high levels. Conical lip seal 200 continues to prevent fluid flow therethrough (thus preventing an unseating of cylinder poppet 22); however, the higher pressures being generated are sufficient to force front face 210 through annulus 205. Thus a compression of pump bulb 18 causes reservoir poppet 20 to move from the deactivated position to the activated position, from which the cylinders are inflated in the above described manner.

Various embodiments have been shown and described to prevent spontaneous inflation. It is to be understood that though these embodiments have been shown and described in isolation, various features of each embodiment can be combined with the others to produce a variety of embodiments.

While the present invention has been described with respect to a pump and valve assembly for a penile implant, the use of generated overpressure to seal a fluid aperture has many other applications within the scope and spirit of the present invention. For example, artificial sphincters utilize fluid pressure to maintain a body cavity or natural passageway in a closed or sealed state. When actuated, fluid pressure is released from the sphincter, causing the bodies' passageway to open. As such, the fluid pressure generated could be used to assist the artificial sphincter in either state. Likewise, many other uses for an overpressure seal exist, both specifically within the field of medical devices and within the field of fluid/gas handling devices in general.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited in the particular embodiments which have been described in detail therein. Rather, reference should be made to the appended claims as indicative of the scope and content of the present invention.

What is claimed is:

1. A pump assembly for an implantable prosthesis, comprising:
a housing having a fluid passageway, the fluid passageway having an inlet and an outlet;
a first flow valve located within the fluid passageway between the inlet and the outlet; and
a supplemental biasing mechanism responsive to inadvertent pressure increases from the inlet to supplement the sealing capabilities of the first flow valve.

2. The pump assembly of claim 1, wherein the supplemental biasing mechanism further includes:
a flexible flap in contact with the first flow valve when the first flow valve is in a closed position so that as fluid pressure from the inlet increases, the flexible flap is caused to seal more firmly against the first flow valve.

3. The pump assembly of claim 2 further comprising:
a tapered passageway for receiving the first flow valve, wherein the flexible flap is part of the tapered passageway.

4. The pump assembly of claim 1, wherein the supplemental biasing mechanism further includes:
a relief area in contact with the first flow valve when the first flow valve is in a closed position, so that as pressure from the inlet increases the relief area expands and further seals against the first flow valve.

5. The pump assembly of claim 1 wherein the supplemental biasing mechanism further includes:
a reservoir chamber disposed within the housing between the inlet and the first flow valve, wherein the reservoir chamber includes an outer wall; and
a portion of the first flow valve which extends into the reservoir chamber and is coupled to the outer wall, so that as fluid pressure within the reservoir chamber increases the outer wall is caused to flex, pulling the flow valve towards a closed position.

6. The pump assembly of claim 5 further including:
a spring coupling the flow valve to the outer wall.

7. The pump assembly of claim 6 wherein the spring is biased to maintain the flow valve in close proximity to the outer wall so that as the fluid pressure increases in the reservoir chamber, the spring will aid in the intentional unseating of the flow valve after a pump bulb compression.

8. The pump assembly of claim 5 further comprising:
a second flow valve, disposed between the first flow valve and the outlet.

9. The pump assembly of claim 5 wherein a tip of the first flow valve is directly connected the outer wall.

10. The pump assembly of claim 9 wherein the tip includes a T-shaped portion that is recessed behind at least one slot in the outer wall.

11. The pump assembly of claim 1, wherein the supplemental biasing mechanism includes:
a spring biasing the first flow valve into a sealed position, wherein the biasing force of the spring is selected to be strong enough to oppose pressures generated in an overpressurization situation within a reservoir and keep the second flow valve in the sealed position.

12. The pump assembly of claim 1 1, further including:
a face coupled to the first valve wherein the face has a large diameter compared to a diameter of the remainder of the first flow valve so that suction forces generated after a compression of a pump bulb act on a sufficient surface area of the face to overcome the biasing force of the spring.

13. The pump assembly of claim 12 further including:
a first lip seal located within the housing for selectively engaging an inner diameter portion of the face in a substantially fluid tight manner; and
a second lip seal located within the housing for selectively engaging an outer diameter portion of the face in a substantially fluid tight manner.

14. The pump assembly of claim 1, wherein the supplemental biasing mechanism further includes:
a front face on the first flow valve for selectively sealing and unsealing an opening to the inlet;
a rear section protruding from the first flow valve away from the opening, including an internal fluid passageway and a throughbore providing access into the internal fluid passageway and an outlet providing an egress from the internal fluid passageway;
a valve sleeve slidably engaging the rear section to selectively seal and unseal the throughbore so that as higher pressure levels are generated within the inlet, front face of the first flow valve is caused to unseal the opening and the valve sleeve is caused to seal the throughbore, wherein the valve sleeve contacts a portion of the housing and prevents fluid flow to the outlet.

15. The pump assembly of claim 1, wherein the supplemental biasing mechanism further includes:
a conical lip seal selectively engageable with a stem portion of the first flow valve, wherein the stem portion includes a cylindrical portion and a groove so that when the groove is positioned adjacent the conical lip seal fluid flow is permitted and when the cylindrical portion is positioned adjacent the conical lip seal fluid flow is prevented in direction from the inlet to the outlet.

16. The pump assembly of claim 15, comprising;
a face forming a portion of the first flow valve, wherein the face is selectively engageable with and biased towards a valve seat; and
an annulus spaced from the valve seat and allowing the face to be forcibly moved therethrough so that the face is retained on a first side or a second side of the annulus.

17. The pump assembly of claim 16, further comprising:
a spacer separating a rear portion of the face from the annulus when said face is positioned between the annulus and the valve seat so that fluid flow is permitted around the rear portion and through the annulus.

18. The pump assembly of claim 17 wherein the spacer is a plurality of bumps located on the annulus.

19. A penile prosthesis comprising:
a housing;
a fluid inlet to the housing, coupleable to a reservoir;
a fluid outlet from the housing, coupleable to an inflatable cylinder;
a fluid passageway coupling the inlet to the outlet;
a first check valve disposed within the fluid passageway and biased towards a closed position;
a second check valve disposed within the fluid passageway and biased towards a closed position;
a pump bulb in fluid communication with the fluid passageway between the first and second check valves; and
a reservoir chamber coupling the inlet to the fluid passageway, wherein a portion of the first check valve extends into the reservoir chamber and is coupled to an outer wall of the reservoir chamber so that as fluid pressure within the reservoir chamber increases, an expansion of the reservoir chamber occurs which urges the first check valve towards a closed position.

20. The prosthesis of claim 19 wherein a negative pressure generated by an expansion of the pump bulb is sufficient to open the first check valve.

21. The pump assembly of claim 19 further including:
a spring coupling the flow valve to the outer wall.

22. The pump assembly of claim 19 wherein the spring is biased to maintain the first check valve in close proximity to the outer wall so that as the fluid pressure increases in the reservoir chamber, the biasing of the spring will aid in the intentional unseating of the flow valve after a pump bulb compression.

23. A method of preventing inadvertent inflation of an implantable prosthetic comprising the steps of:
biasing a valve assembly such that an outlet is substantially closed; and
using inadvertent pressure increases from the inlet to supplement the biasing of the valve assembly.

24. The method of claim 23, wherein the step of using inadvertent pressure includes:
preventing fluid flow through the outlet by selectively varying fluid pressure within a bypass passageway having a first end which is in fluid communication with an inlet and a second end which is in fluid communication with a chamber.

25. The method of claim 24, further comprising the steps of:
displacing a flexible abutting wall disposed between the chamber and the valve assembly so that the abutting wall is caused to contact the valve assembly and urge the valve assembly into a closed position when the fluid pressure within the chamber exceeds a predetermined amount.

26. The method of claim 24, further comprising the steps of:
sliding a valve sleeve along a rear portion of the valve assembly to occlude a passageway leading through a portion of the valve assembly and to sealingly engage a portion of housing.

27. A method of preventing inadvertent inflation of an implantable prosthetic comprising the steps of:
biasing a valve assembly such that an outlet is substantially closed, wherein a biasing mechanism is sufficiently strong to oppose increased pressure levels generated during an overpressurization situation;
providing a sufficient surface area on the valve assembly so that vacuum forces generated after a compression of a pump bulb are sufficient to open the valve assembly.

28. A pressure lock out arrangement for an inflatable prosthesis comprising:
a housing having an inlet and an outlet;
a valve disposed between the inlet and the outlet, the valve being biased toward substantially sealing the outlet; and
a supplemental biasing mechanism responsive to inadvertent pressure increases from the inlet to increase the biasing of the valve toward substantially sealing the outlet.

29. The pressure lock out of claim 28 wherein the supplemental biasing mechanism further comprises:
a front face on the valve for selectively sealing and unsealing an opening to the inlet;
a rear section protruding from the valve away from the opening, including an internal fluid passageway and a throughbore providing access into the internal fluid passageway and an outlet providing an egress from the internal fluid passageway;
a valve sleeve slidably engaging the rear section to selectively seal and unseal the throughbore so that as higher pressure levels are generated within the inlet, front face of the valve is caused to unseal the opening and the valve sleeve is caused to seal the throughbore, wherein the valve sleeve contacts a portion of the housing and prevents fluid flow to the outlet.

30. The pressure lock out of claim 28 wherein the supplemental biasing mechanism further comprises:
a front face on the valve for selectively sealing and unsealing an opening to the inlet;
a rear section protruding from the valve away from the opening, including an external fluid passageway having an outlet end;
a valve slidably engaging the rear section to selectively seal and unseal the outlet end so that as higher pressure levels are generated within the inlet, the front face of the valve is caused to unseal the opening and the valve sleeve is caused to seal the outlet end, wherein the valve sleeve contacts a portion of the housing and prevents fluid flow to the outlet end.

31. The pressure lock out of claim 28 wherein the supplemental biasing mechanism further comprises:
a reservoir chamber disposed within the housing between the inlet and the valve, wherein the reservoir chamber includes an outer wall; and
a portion of the flow valve which extends into the reservoir chamber and is coupled to the outer wall, so that as fluid pressure within the reservoir chamber increases the outer wall is caused to flex, pulling the flow valve towards a closed position.

32. The pressure lock out of claim 28 further including:
a spring coupling the flow valve to the outer wall.

33. The pressure lock out of claim 28, wherein the supplemental biasing mechanism further includes:
a conical lip seal selectively engageable with a stem portion of the valve, wherein the stem portion includes a cylindrical portion and a groove so that when the groove is positioned adjacent the conical lip seal fluid flow is permitted and when the cylindrical portion is positioned adjacent the conical lip seal fluid flow is prevented in direction from the inlet to the outlet.

34. The pressure lock out of claim 33, comprising;
a face forming a portion of the valve, wherein the face is selectively engageable with and biased towards a valve seat; and
an annulus spaced from the valve seat and allowing the face to be forcibly moved therethrough so that the face is retained on a first side or a second side of the annulus.

35. The pressure lock out of claim 34, further comprising:
a spacer separating a rear portion of the face from the annulus when said face is positioned between the annulus and the valve seat so that fluid flow is permitted around the rear portion and through the annulus.

36. The pressure lock out of claim 35 wherein the spacer is a plurality of bumps located on the annulus.

37. A penile prosthesis comprising:
a housing;
a fluid inlet to the housing, coupleable to a reservoir;
a fluid outlet from the housing, coupleable to an inflatable cylinder;
a reservoir chamber disposed within the housing and fluidly coupled to the inlet;
a fluid passageway fluidly coupled to the reservoir chamber;
a first check valve disposed within the fluid passageway and biased towards a closed position;
a second check valve disposed within the fluid passageway and biased towards a closed position;
a pump bulb in fluid communication with the fluid passageway between the first and second check valves;
a spring biasing the first check valve into a sealed position, wherein the biasing force of the spring is selected to be strong enough to oppose pressures generated in an overpressurization situation and keep the second check valve in the sealed position;
a face coupled to the first check valve wherein the face has a large diameter compared to a diameter of the remainder of the flow valve so that suction forces generated after a compression of a pump bulb act on a sufficient surface area of the face to overcome the biasing force of the spring

38. The pump assembly of claim 37 further including:
a first lip seal located within the housing for selectively engaging an inner diameter portion of the face in a substantially fluid tight manner; and
a second lip seal located within the housing for selectively engaging an outer diameter portion of the face in a substantially fluid tight manner.

39. A penile prosthesis comprising:
a housing;
a fluid inlet to the housing, coupleable to a reservoir;
a fluid outlet from the housing, coupleable to an inflatable cylinder;
a reservoir chamber disposed within the housing and fluidly coupled to the inlet;
a fluid passageway fluidly coupled to the reservoir chamber;
a first check valve disposed within the fluid passageway and biased towards a closed position;
a second check valve disposed within the fluid passageway and biased towards a closed position;
a pump bulb in fluid communication with the fluid passageway between the first and second check valves;
a front face on the first check valve for selectively sealing and unsealing an opening to the fluid inlet;
a rear section protruding from the first check valve away from the opening, including an internal fluid passageway and a throughbore providing access into the internal fluid passageway and an outlet providing an egress from the internal fluid passageway;
a valve sleeve slidably engaging the rear section to selectively seal and unseal the throughbore so that as higher pressure levels are generated within the inlet, front face of the first check valve is caused to unseal the opening and the valve sleeve is caused to seal the throughbore, wherein the valve sleeve contacts a portion of the housing and prevents fluid flow to the outlet.

40. A penile prosthesis comprising:
a housing;
a fluid inlet to the housing, coupleable to a reservoir;
a fluid outlet from the housing, coupleable to an inflatable cylinder;
a reservoir chamber disposed within the housing and fluidly coupled to the inlet;
a fluid passageway fluidly coupled to the reservoir chamber;
a second check valve disposed within the fluid passageway and biased towards a closed position;
a first check valve disposed within the fluid passageway and biased towards a closed position, the first check valve having a front face for selectively sealing and unsealing an opening to the fluid inlet and a rear stem section protruding from the first check valve away from the opening, including a cylindrical portion and a groove;
a pump bulb in fluid communication with the fluid passageway between the first and second check valves;
a conical lip seal integral with the housing and positioned so that the rear stem section is moveable therein so that when a cylindrical portion of the stem section is aligned with the conical lip seal, fluid flow in a direction from the inlet to the outlet is prevented and increased pressure levels within the inlet serves to further seal the conical lip seal against the cylindrical portion of the stem section, and when the groove is aligned with the conical lip seal, fluid flow is permitted; and
an annulus integral with the housing positioned so as to cooperate with the front face, allowing the front face to be forcibly move therethrough so that when the front face is on a first side of the annulus, the groove of the stem section is aligned with the conical lip seal and when the front face is on a second side of the annulus the cylindrical portion of the stem is aligned with the conical lip seal.

41. The penile prosthesis of claim 40, further comprising:
a spacer separating a rear portion of the front face from the annulus when said front face is on said first side so that fluid flow is permitted around the rear portion and through the annulus.

42. The penile prosthesis of claim 41 wherein the spacer is a plurality of bumps located on the annulus.

43. A method of preventing a vacuum lock from occurring in a penile prosthesis having a valve movable through an annulus so that when on a first side of the annulus operation of the prosthesis is permitted and when on a second side, spontaneous inflation is prevented, comprising:
positioning the valve on the first side of the annulus;
providing a fluid path around the valve through the annulus when a rear face of the valve is proximate the annulus.

44. The method of claim 43 wherein the step of providing includes:
providing at least one spacer to prevent the rear face from sealing against he annulus.

45. The method of claim 44 wherein the at least one spacer is integral with the annulus.
